(19) United States
(12) Patent Application Publication
(10) Pub. No.: US 2013/0072733 A1
(43) Pub. Date: Mar. 21, 2013

(54) METHOD OF SEPARATING PENTENE-2 FROM A C5 CUT CONTAINING PENTENE-2 AND PENTENE-1 BY SELECTIVE OLIGOMERIZATION OF PENTENE-1

(71) Applicant: IFP Energies Nouvelles, Rueil- Malmaison Cedex (FR)

(72) Inventors: Pierre-Alain Breuil, Lyon (FR); Helene Olivier-Bourbigou, Saint Genis-Laval (FR); Adrien Boudier, Lyon (FR)

(73) Assignee: IFP ENERGIES NOUVELLES, Rueil-Malmaison Cedex (FR)

(21) Appl. No.: 13/622,610

(22) Filed: Sep. 19, 2012

(30) Foreign Application Priority Data
Sep. 20, 2011 (FR) 11/02.847

(51) Int. Cl. C07C 7/177; C07C 7/152; C07C 7/163

(52) U.S. Cl. 585/264; 585/809; 585/850

(57) ABSTRACT

The present invention describes a method of separating pentene-2 from a C5 cut containing pentene-2 and pentene-1 by selective oligomerization of pentene-1 to dimers having a branching index less than or equal to 1. Optional additional steps make it possible to separate 2-methyl-2-butene, 2-methyl-1-butene, n-pentane, iso-pentane, pentadienes as well as traces of acetylene hydrocarbons optionally present in the C5 feedstock.

The present invention describes a method of separating pentene-2 from a C5 cut containing pentene-2 and pentene-1 by selective oligomerization of pentene-1 to dimers having a branching index less than or equal to 1 in the presence of an iron-based catalytic composition. Optional additional steps make it possible to separate other compounds typically present in C5 cuts.

C5 cuts are available in large quantities in refineries and petrochemical plants. They are produced by the standard processes such as catalytic cracking or steam cracking. These C5 cuts typically contain olefinic compounds such as linear pentenes (pentene-1 and pentenes-2) and branched pentenes (such as the isoamylenes: 2-methyl-1-butene and 2-methyl-2-butene), and optionally paraffins (isopentane and n-pentane), dienes (such as isoprene) as well as acetylenic impurities. Table 1 shows the typical compositions of C5 cuts originating from catalytic cracking or from steam cracking.

TABLE 1
Typical compositions of C5 cuts

| | Typical composition | Composition |
|---|---|---|
| | (wt. %) | (wt. %) |
| | catalytic cracking | steam cracking |
| C4- | 2 | 1 |
| n-pentane | 5.5 | 26.0 |
| isopentane | 31.5 | 24.0 |
| n-pentenes | 22.5 | 4.5 |
| methylbutenes | 37.5 | 12.0 |
| cyclopentene | — | 1.5 |
| isoprene | traces | 13.5 |
| pentadiene | — | 9.0 |
| (piperylene) | | |
| cyclopentadiene | — | 7.5 |
| butyne-2 | — | traces |
| C6+ | 1.0 | 1.0 |

The C5 cuts offer high contents of olefins and of branched constituents, which gives them high octane numbers (Table 2). Furthermore, they are necessary for obtaining a regular distillation curve of commercial fuel bases. Accordingly, they are not generally separated but are distilled with the aromatic hydrocarbons to be sent directly to the preparation of fuels. Only isoprene and cyclopentadiene are distilled on an industrial scale. The isoamylenes are often extracted from the C5 cut by an etherification reaction to form alkyl tert-amyl ethers, which are components with a high octane number.

Other developments allow more extensive separations of the C5 cut. Thus, patent EP0869107 describes a method of separating alpha-olefin, tertiary olefin and/or ether from an unsaturated hydrocarbon cut comprising a C5 cut. Patent FR1540692 describes a method of production of iso-olefins of high purity, including isobutene and 2-methyl-2-butene. It consists of absorbing the iso-olefin with a solution of sulphuric acid in order to separate it and then heating and separating the iso-olefin with steam. Patents FR2871167 and FR2871168 describe processes for production of a diesel fuel cut from gasoline cuts comprising a first step of separation of the normal and iso-olefins.

Whatever process is used, their separation is still often problematic. Given the small difference in boiling points of these compounds (see Table 2), it is difficult to separate them by distillation, and in particular pentene-1 and 2-methyl-1-butene, as well as 2-methyl-2-butene and pentenes-2, cannot be separated by distillation. This is why more effective and selective physical and chemical separation techniques must be implemented.

TABLE 2
Boiling point of the C5 compounds:

| | Boiling point (° C.) | RON/MON |
|---|---|---|
| n-Pentane | 35-36 | 62.0/61.9 |
| Pentene-1 | 30 | 99.9/77.1 |
| Pentene-2 Cis | 37-38 | 98.9/80.0 |
| Pentene-2 Trans | 37 | 98.9/80.0 |
| 2-Methyl-1-butene | 31 | 102.5/81.9 |
| 2-Methyl-2-butene | 35-38 | 97.3/84.7 |

Furthermore, there are methods for the oligomerization of linear alpha-olefins. Patent US2002/0177744 describes the synthesis of butene-1 dimers and other linear alpha-olefins by coupling of alpha-olefins catalyzed by iron/cobalt complexes activated with an aluminium derivative. The dimers formed have the particular feature that they have very good linearity.

It has now been found that these iron complexes could be used for selectively converting pentene-1 by an oligomerization reaction when the latter is mixed with a feedstock containing pentene-2 and optionally 2-methyl-1-butene, 2-methyl-2-butene, n-pentane and/or isopentane.

The oligomerization of light olefins (C2-C5) by transition metal complexes is mainly described in the literature in the case of ethylene. It is in fact well known that the reactivity of the olefins decreases as the length of their carbon chain increases (C2>>C3>C4>5). The best-known systems for converting butenes by oligomerization are nickel-based systems of the Ziegler type. These systems convert butene-1 and butene-2 to a mixture of octenes (for reference see “Reaction of unsaturated compounds”, p 240-253 in “Applied Homogeneous Catalysis with Organometallics” Ed. B. Cornils, W. Herrmann, Wiley-VCH, 2002).

The iron-based systems according to the invention are therefore different from the systems known from the literature. It is in fact surprising that this catalytic system reacts a little, if at all, with pentene-2.

The objective of the present invention is therefore to propose a method of separating pentene-2 from a C5 feedstock comprising pentene-1 and pentene-2, characterized by the following successive steps:
a step of selective oligomerization of pentene-1 to oligomers having a branching index less than or equal to 1 by an iron-based catalytic composition
a step of separation of pentene-2.

The method according to the invention thus allows, on the one hand, the effective separation of pentene-2 from pentene-1 and, on the other hand, the simultaneous production of oligomers (mainly decenes) having a branching index less than or equal to 1.

The branching index is calculated as follows: ((wt. % n-olefins)×0+(wt. % monobranched i-olefins)×1+(wt. % dibranched i-olefins)×2 . . . ))/100. A branching index less than or equal to 1 indicates that the oligomers formed are predominantly linear and monobranched.

The dimers having a branching index less than or equal to 1 are used in fuel pools or as raw material in a cracking step for propylene production or as intermediates in petrochemistry, for example for the synthesis of alcohols by hydroformylation and hydrogenation.

Other separation steps can be added to the oligomerization step in the case of a feedstock containing, in addition to pentene-1 and pentene-2, other C5 components such as 2-methyl-2-butene, 2-methyl-1-butene, iso-pentane, n-pentane, pentadienes as well as traces of acetylene hydrocarbons optionally present in the C5 feedstock.

This succession of steps offers numerous advantages and makes it possible to produce products that can be used in numerous applications.

Thus, a step of separation of 2-methyl-2-butene and 2-methyl-1-butene is preferably carried out before the step of selective oligomerization by etherification in order to produce alkyl tert-butyl ethers.

Etherification makes it possible to produce alkyl tert-amyl ethers (of the TAME type), which are sought as components with a high octane number.

In the case where the feedstock contains pentadienes and traces of acetylene hydrocarbons, a step of separation of the pentadienes and traces of acetylene hydrocarbons by selective hydrogenation is preferably carried out before the step of separation of 2-methyl-2-butene and 2-methyl-1-butene by etherification (optional) and/or before the step of selective oligomerization.

The most reactive compounds in the cut, namely the pentadienes and traces of acetylene hydrocarbons, are thus converted in the very first step, and therefore will not cause parasitic reactions in the subsequent steps. Moreover, selective hydroisomerization of pentadienes makes it possible to increase the concentration of pentene-2, promoting the yield of this sought product.

Similarly, a step of co-oligomerization of the predominantly linear oligomers obtained by selective oligomerization with pentene-2 in the presence of an acid catalyst can be carried out after the step of selective oligomerization. This step makes it possible to obtain pentadecenes. The pentadecenes can be introduced into a kerosene cut or can be used as solvent.

The co-oligomerization effluent containing the pentadecenes can be subjected to a step of separation, for example by distillation, making it possible to separate the n-pentane, if present.

### DETAILED DESCRIPTION

The Feedstock

The feedstock used is a C5 hydrocarbon cut containing pentene-1 and pentene-2. Typically, besides the pentenes (pentene-1 and pentenes-2), it contains 2-methyl-2-butene, 2-methyl-1-butene, paraffins (isopentanes and n-pentane), pentadienes and traces of acetylene hydrocarbons. These feedstocks can originate from a catalytic cracking unit, for example of the fluidized bed type, and/or from a unit for steam cracking (of naphtha) and/or from any other sources.

Depending on the various components of the feedstock, one or more separating steps can advantageously precede or follow the step of selective oligomerization of pentene-1. The various embodiments will be described below.

The method of separating pentene-2 from a C5 feedstock containing pentene-1, pentene-2 and optionally 2-methyl-2-butene, 2-methyl-1-butene, iso-pentane, n-pentane, pentadienes as well as traces of acetylene hydrocarbons, advantageously comprises the following succession of steps:
an optional step of selective hydrogenation of the pentadienes and acetylenic impurities,
an optional step of separation of 2-methyl-2-butene and 2-methyl-1-butene by etherification,
a step of selective oligomerization of pentene-1 to dimers having a branching index less than or equal to 1,
a step of separation of the oligomers formed from pentene-2,
an optional step of co-oligomerization of the predominantly linear oligomers with pentene-2 to produce pentadecenes,
then an optional step of separation of the n-pentane and iso-pentane.

Selective Hydrogenation (Optional)

The feedstock containing pentadienes and traces of acetylene hydrocarbons, in addition to pentene-1 and pentene-2, can be subjected to a step of selective hydrogenation.

In this step, the pentadienes are selectively hydrogenated to mono-olefins and the alpha-olefins are isomerized in thermodynamic equilibrium to internal olefins, i.e. to pentene-2 and 2-methyl-2-butene. This step also makes it possible to remove the traces of acetylene hydrocarbons that are always present in these cuts and which are poisons or contaminants for the subsequent steps. In fact, this step makes it possible to convert the most reactive compounds in the cut, namely the pentadienes and the acetylenic compounds in the very first step, and therefore they will not cause parasitic reactions in the subsequent steps. This reaction thus makes it possible to increase the yield of n-pentenes.

In this first optional step of selective hydrogenation, the following reactions are therefore carried out simultaneously:
selective hydroisomerization of the pentadienes to a mixture of n-pentenes in thermodynamic equilibrium,
isomerization of the alpha-olefins to internal olefins (pentene-1 to pentene-2, 2-methyl-1-butene to 2-methyl-2-butene), also in thermodynamic equilibrium,
hydrogenation of the traces of acetylene hydrocarbons.

In the case of a feedstock originating from a steam cracker, a step of selective hydrogenation of the dienes is preferably carried out. In the case of a feedstock originating from a catalytic cracking unit, for example of the fluidized bed type, the step of selective hydrogenation of the dienes is optional. If the concentration of diolefins or acetylene in the cut used for said method is greater than 1000 ppm, selective hydrogenation is recommended in order to reduce this concentration to below 1000 ppm. Feedstocks containing more than 300 ppm of these impurities or even more than 10 ppm will preferably be treated in this step.

The reactions can be carried out by means of various specific catalysts comprising one or more metals, for example from group 10 of the periodic table (Ni, Pd, Pt), deposited on a support. Preferably a catalyst is used that comprises at least one palladium compound fixed on a refractory mineral support, for example on an alumina. The palladium content on the support can be comprised between 0.01 and 5 wt. %, preferably between 0.05 and 1 wt. %. Various forms of pretreatment known to a person skilled in the art can optionally be applied to these catalysts in order to improve selectivity in the hydrogenation of pentadienes to pentenes at the expense of total hydrogenation to pentane, which must be avoided. The catalyst preferably contains 0.05 to 10 wt. % of sulphur. Advantageously, a catalyst constituted by palladium deposited on alumina and containing sulphur is used.

The sulphurization of the catalyst can be carried out in situ (in the reaction zone) or preferably ex situ. In the latter case, the catalyst has advantageously been treated, before being loaded into the hydrogenation reactor, with at least one sulphur-containing compound diluted in a solvent. The catalyst obtained, containing 0.05 to 10 wt. % of sulphur, is loaded into the reactor and activated under neutral or reducing atmosphere at a temperature comprised between 20 and 300° C., a pressure comprised between 0.1 and 5 MPa and an LHSV comprised between 50 and 600 h⁻¹, and the feedstock is brought into contact with said activated catalyst.

The utilization of a catalyst, preferably a palladium catalyst, is not critical, but it is generally preferable to use at least one reactor with descending flow through a fixed catalyst bed. When there is a high proportion of pentadienes in the cut, which is the case for example with a cut from steam cracking when extraction of the pentadienes for specific uses is not desired, it may be advantageous to carry out the conversion in two reactors in series, for better control of hydrogenation selectivity. The second reactor can have ascending flow and can have a finishing role.

The quantity of hydrogen required for all of the reactions carried out in this step is adjusted depending on the composition of the cut, so that there is advantageously only a slight excess of hydrogen relative to the theoretical stoichiometry.

The operating conditions are chosen in such a way that the reagents and the products are in the liquid phase. It may, however, be advantageous to choose an operating mode such that the products are partially vaporized at the reactor outlet, which facilitates thermal control of the reaction. The temperature can vary from 20 to 200° C., preferably from 50 to 150° C. or more preferably from 60 to 150° C. The pressure can be adjusted between 0.1 and 5 MPa, preferably between 0.5 and 4 MPa and advantageously between 0.5 and 3 MPa, in such a way that the reagents, at least partly, are in the liquid phase. The space velocity can be comprised between 0.5 and 10 h⁻¹ and preferably between 1 and 6 h⁻¹, with an H₂/diolefins (molar) ratio from 0.5 to 5 and preferably from 1 to 3.

Preferably, the selective hydrogenation of the pentadienes and traces of acetylene hydrocarbons is carried out with a catalyst comprising at least one metal chosen from the group formed by nickel, palladium and platinum, deposited on a support, at a temperature of 20-200° C., a pressure of 1-5 MPa, a space velocity of 0.5-10 h⁻¹ and with an H₂/pentadienes (molar) ratio from 0.5 to 5.

The hydroisomerization reactor or reactors can advantageously be followed by a stabilizing column, which removes the excess traces of hydrogen and any methane.

Etherification of 2-Methyl-2-Butene and 2-Methyl-1-Butene (Optional)

The feedstock containing, in addition to pentene-1 and pentene-2, isoamylenes (2-methyl-2-butene and 2-methyl-1-butene), and preferably after removing the pentadienes and traces of acetylene hydrocarbons, can be subjected to an etherification of the isoamylenes in order to form a tertiary alkyl ether by contacting in a reaction zone containing an etherification catalyst. The etherification step is known to a person skilled in the art.

This optional step preferably takes place after the optional step of selective hydrogenation but before the step of selective oligomerization.

The aim of the second optional step is to convert the 2-methyl-2-butene and 2-methyl-1-butene present in the C5 cut to an alkyl tert-amyl ether by etherification in the presence of an etherification catalyst with an alcohol the hydrocarbon chain of which can comprise from 1 to 10 carbon atoms. Methanol is preferably used as the alcohol for producing the TAME (tert-amyl methyl ether).

The conversion is carried out by means of an acid catalyst, for example a catalyst based on an ion exchange resin in the H⁺form, such as a sulphonic resin —SO₃H. This catalyst can be used for example conventionally in a fixed bed, or in a moving bed. It is preferable to work with an expanded bed of catalyst, maintained by circulation of the reaction mixture in ascending flow through the reactor and an external heat exchanger. This manner of operating makes it possible to monitor the functioning of the catalyst easily as well as remove the heat produced in the reaction, avoiding the formation of hot spots.

A finishing reactor can advantageously be installed after the expanded-bed reactor for maximum exhaustion of the isoamylenes in the residual cut and to increase the yield of ether, but the major part of the reaction takes place in the expanded-bed reactor. Maximum exhaustion of the isoamylenes can also be achieved in the finishing process, by using reactive distillation (distillation column containing catalyst), which makes it possible to increase the conversion by means of separation of the products in the reactor.

The operating conditions are chosen in such a way that the reagents and the products are in the liquid phase. The temperature is generally set so that the reaction takes place at a sufficient rate, for example from 30 to 130° C., preferably from 40 to 100° C., and the pressure is consequently adjusted in such a way that the reagents are in the liquid phase.

Fractionation of the product originating from the etherification step makes it possible to obtain, on the one hand, an organic fraction enriched in tertiary alkyl ether and on the other hand an organic fraction depleted of tertiary alkyl ether containing pentene-1, pentene-2 and optionally the pentanes, if present. Thus, the reaction section is followed by a distillation section, where the ether at the bottom of the column and a distillate comprising the residual C5 cut and the excess alcohol as well as traces of other volatile oxygen-containing compounds are separated. The alcohol is separated by washing with water and the water-alcohol mixture is distilled in order to recycle the alcohol.

The TAME (tert-amyl methyl ether) thus produced is generally used as an additive for increasing the octane number in reformulated gasolines. It is also possible to recover the isoamylenes from the ethers produced, which are easily decomposed in the presence of an acid catalyst to release the purified isoamylenes.

The residual C5 cut thus obtained still contains pentene-2 and pentene-1 as well as optionally pentanes (iso and normal). It is subjected to the step of selective oligomerization.

Selective Oligomerization

The feedstock containing pentene-1 and pentene-2, and preferably after removal of the pentadienes and traces of acetylene hydrocarbons and optionally after removal of the isoamylenes, is subjected to a step of selective oligomerization of pentene-1 by iron-based catalytic compositions without significant reaction of the pentene-2. This step leads mainly to the formation of decenes having a branching index less than or equal to 1. In the case of a feedstock containing C5s, the selectivity for dimers (decenes) is generally greater than 50%, preferably greater than 80% relative to the converted olefins. The selectivity for dimers provides a branching index less than or equal to 1 and preferably less than 0.6.

The iron-based catalytic composition used in the step of selective oligomerization is composed of at least one iron precursor, at least one ligand of the formula described below, complexed or not complexed with the iron precursor, and optionally an activating agent.

The iron precursor has the general formula FeXₙ, X being an anionic group, for example a halide such as a chloride, a fluoride, a bromide or an iodide; or a hydrocarbon group, for example a methyl, a benzyl or a phenyl; a carboxylate, for example an acetate or an acetylacetonate; an oxide; an amide, for example a diethyl amide; an alkoxide, for example a methoxide, an ethoxide or a phenoxide; or a hydroxyl. Alternatively, X can be a non-coordinating or weakly coordinating anion, for example a tetrafluoroborate, a fluorinated aryl borate or a triflate. The anionic group X can be monoanionic or dianionic. The iron precursors can be hydrated or not, coordinated or not with a ligand.

By way of example, the iron precursors can be iron(II) chloride, iron(III) chloride, iron(II) fluoride, iron(III) fluoride, iron(II) bromide, iron(III) bromide, iron(II) iodide, iron(III) iodide, iron(II) acetate, iron(III) acetate, iron(II) acetylacetonate, iron(III) acetylacetonate, iron(II) octoate, iron(III) octoate, iron(II) 2-ethylhexanoate, iron(III) 2-ethylhexanoate, iron(II) triflate, iron(III) triflate, iron(III) nitrate. The iron precursors can be hydrated or not, coordinated or not with a ligand such as tetrahydrofuran for example.

The ligand has the general formula:

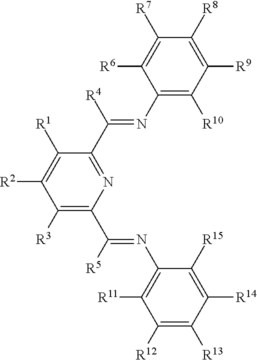

where R¹, R², R³, R⁴, R⁵, R⁶, R⁷, R⁸, R⁹, R¹⁰, R¹¹, R¹², R¹³, R₁₄ and R¹⁵, which may be identical or different, are chosen from the hydrogen atom, linear or branched, cyclic or non-cyclic, saturated or unsaturated alkyl groups, aryl, aralkyl or alkaryl groups comprising 1 to 12 carbon atoms, groups containing hetero-elements, heterocyclic or not, aromatic or not, halides or not, supported or not, and preferably chosen from the alkoxy, nitro, halides and/or perfluoroalkyl group.

As non-limitative examples, R¹, R², R³, R⁴, R⁵, R⁶, R⁷, R⁸, R⁹, R¹⁰, R¹¹, R¹², R¹³, R¹⁴ and R¹⁵ can be chosen from hydrogen, methyl, ethyl, isopropyl, isobutyl, tert-butyl, cyclohexyl, phenyl, benzyl, methoxy, nitro, dimethylamine, diethylamine, diisopropylamine, trifluoromethyl, fluoride, chloride, bromide and iodide groups.

By way of example, the ligands can be of the following formula:

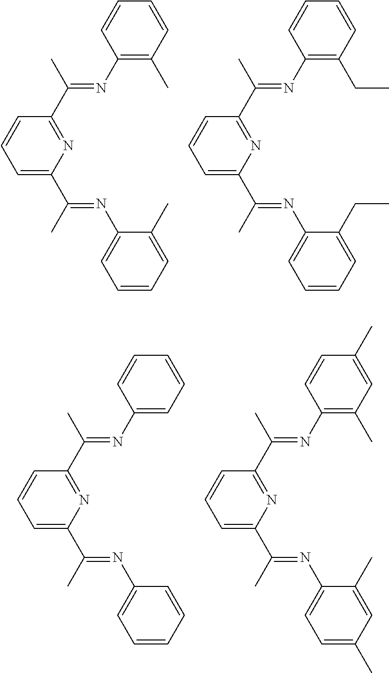 [SMILES: C/C(=N\c1ccccc1C)c1cccc(/C(C)=N/c2ccccc2C)n1 ; CCc1ccccc1/N=C(\C)c1cccc(/C(C)=N/c2ccccc2CC)n1 ; C/C(=N\c1ccccc1)c1cccc(/C(C)=N/c2ccccc2)n1 ; C/C(=N\c1ccc(C)cc1C)c1cccc(/C(C)=N/c2ccc(C)cc2C)n1]

By way of example, the complexes can be of the following formula:

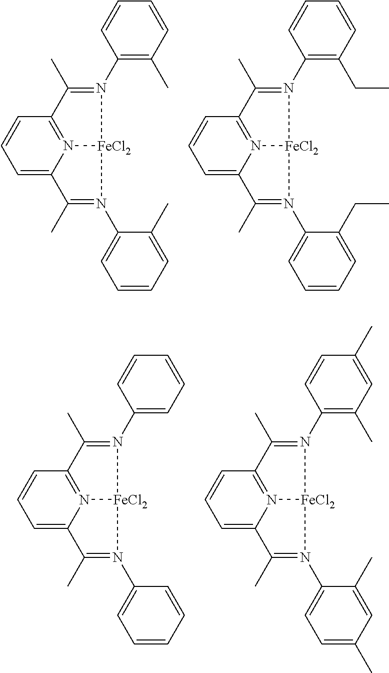

The optional activator for the catalyst used in the present invention is preferably a Lewis acid. Preferably, the Lewis acid is chosen from aluminium derivatives and boron or zinc derivatives or a mixture of these derivatives.

Examples of aluminium derivatives can include alkylaluminiums, for example trimethylaluminium, triethylaluminium, tributylaluminium, tri-n-octylaluminium; alkylaluminium halides, for example diethylaluminium chloride, ethylaluminium dichloride, ethylaluminium sesquichloride, methylaluminium dichloride, isobutylaluminium dichloride; and aluminoxanes. The aluminoxanes are well known to a person skilled in the art as oligomeric compounds that can be prepared by controlled addition of water to an alkylaluminium, for example trimethylaluminium. Such compounds can be linear, cyclic or mixtures of these compounds. They are generally represented by the formula [RAIO]ₐ where R is a hydrocarbon group and “a” is a number from 2 to 50. Preferably, the aluminoxane is chosen from methylaluminoxane (MAO) and/or ethylaluminoxane (EAO) and/or from modified aluminoxanes such as modified methylaluminoxane (MMAO).

Examples of boron derivatives can include the trialkylboranes, such as for example trimethylborane, triethylborane, tripropylborane, tri-n-butylborane, tri-isobutylborane, tri-n-hexylborane, tri-n-octylborane, used alone or in a mixture, tris(aryl)boranes, for example tris(perfluorophenyl)borane, tris(3,5-bis(trifluoromethyl)phenyl)borane, tris(2,3,4,6-tetrafluorophenyl)borane, tris(perfluoronaphthyl)borane, tris(perfluorobiphenyl)borane and derivatives thereof. It is also possible to use, as activator, (aryl)borates associated with a triphenylcarbenium cation or with a trisubstituted ammonium cation such as triphenylcarbenium tetrakis(perfluorophenyl)borate, N,N-dimethylanilinium tetrakis(perfluorophenyl)borate, N,N-diethylanilinium tetrakis(3,5-bis(trifluoromethyl)phenyl)borate, triphenylcarbenium tetrakis(3,5-bis(trifluoromethyl)phenyl)borate.

Examples of zinc derivatives can include the dialkylzincs, such as for example dimethylzinc, diethylzinc, dipropylzinc, dibutylzinc, dineopentylzinc, di(trimethylsilylmethyl)zinc, used alone or in a mixture.

The selective oligomerization of butene-1 can be carried out in the presence of a solvent. The organic solvent used in the catalytic composition will preferably be an aprotic solvent. Among the solvents that can be used in the method according to the present invention, aliphatic or cyclic hydrocarbons such as pentane, hexane, cyclohexane or heptane, aromatic hydrocarbons such as benzene, toluene or xylenes, chlorinated solvents such as dichloromethane or chlorobenzene, or acetonitrile, diethyl ether, tetrahydrofuran (THF) may be mentioned. The organic solvent will preferably be a saturated or unsaturated aliphatic solvent or an aromatic hydrocarbon.

The solvent can also be an ionic liquid, in a mixture or not with an organic solvent. The reaction of selective oligomerization of pentene-1 can be carried out in a closed system, in a semi-open system or continuously, with one or more reaction steps. Vigorous stirring must ensure good contact between the reagent or reagents and the catalytic composition.

The reaction temperature can be from −40 to +250° C., preferably from 0° C. to +150° C. The heat generated by the reaction can be removed by any means known to a person skilled in the art.

The reaction is preferably carried out in the liquid phase, and the pressure is adjusted to maintain the system in the liquid phase. In general, the pressure can vary from atmospheric pressure to 10 MPa; the pressure is preferably comprised between 5 and 8 MPa.

Separation of Pentene-2

The effluent from selective oligomerization thus comprises the oligomers derived from pentene-1, in particular decenes having a branching index less than or equal to 1, pentene-2 and optionally pentanes, if present. This effluent is then subjected to a step of separation, for example distillation, in order to separate the oligomers formed from the pentene-2 (and from the pentanes if present). As the pentanes are inert, an additional separation is not generally carried out.

Co-Oligomerization of Predominantly Linear Oligomers and Pentene-2 (Optional)

In order to upgrade the pentene-2 to a product with higher added value, a step of co-oligomerization of the decenes having a branching index less than or equal to 1 obtained by oligomerization of pentene-1 with pentene-2 can be carried out in order to produce pentadecenes. The pentadecenes thus produced can be introduced into a kerosene cut or used as a solvent.

The catalyst and the operating conditions are chosen in such a way that the reaction is predominantly a dimerization reaction (i.e. an oligomerization or addition reaction limited to two basic molecules). The reaction is considered to be predominantly a dimerization if at least 50%, preferably at least 65% and even more preferably at least 80% of the products obtained are dimers, the remaining percentages consisting of unreacted starting products and products of trimerization or of a higher degree of oligomerization. Similarly, the secondary back cracking reactions are limited by the choice of catalyst and operating conditions.

The catalyst used in the co-oligomerization reaction is an acid catalyst, preferably an amorphous acid catalyst or a catalyst of the zeolite type with an Si/Al ratio greater than 5, preferably comprised between 8 and 80 and even more preferably between 15 and 70. The zeolites are at least partly, preferably practically completely, in acid form, i.e. in hydrogen form (also called proton form). Preferably, the catalyst used is a zeolitic catalyst chosen from the group comprising the zeolites having 8-MR, 10-MR and/or 12-MR channels.

Examples of said preferred zeolites are the zeolites of structures: MEL, MFI, ITH, NES, EUO, ERI, FER, CHA, MFS, MWW, MTT, TON. Among the zeolites of the MEL structural type, zeolite ZSM-11 is preferred. Among the zeolites of the MFI structural type, zeolite ZSM-5 is preferred. Among the zeolites of the ITH structural type, zeolite ITQ13 is preferred. Among the zeolites of the NES structural type, zeolite NU-87 is preferred. Among the zeolites of the EUO structural type, zeolite EU-1 is preferred. Among the zeolites of the ERI structural type, the zeolite erionite is preferred. Among the zeolites of the FER structural type, the zeolites ferrierite and ZSM-35 are preferred. Among the zeolites of the CHA structural type, the zeolite chabazite is preferred. Among the zeolites of the MFS structural type, zeolite ZSM-57 is preferred. Among the zeolites of the MWW structural type, zeolite MCM-22 is preferred. Among the zeolites of the MTT structural type, zeolite ZSM23 is preferred. Among the zeolites of the TON structural type, zeolite ZSM-22 is preferred. These zeolites can be used alone or in a mixture. The zeolites 12MR preferred for this invention are the zeolites of the following structures: MOR, FAU, BEA, BOG, LTL, OFF. Among the zeolites of the MOR structural type, the zeolite mordenite is preferred. Among the zeolites of the FAU structural type, zeolite Y is preferred. Among the zeolites of the BEA structural type, zeolite beta is preferred. Among the zeolites of the BOG structural type, the zeolite boggsite is preferred. Among the zeolites of the LTL structural type, zeolite L is preferred. Among the zeolites of the OFF structural type, the zeolite off retite is preferred. These zeolites can be used alone or in a mixture. The catalyst of the present invention also contains at least one amorphous or poorly crystallized porous mineral matrix of the oxide type and optionally a binder. By way of non-limitative examples of matrices, alumina, silica, silica-alumina may be mentioned. The clays (chosen for example from the natural clays such as kaolin or bentonite), magnesia, titanium oxide, boron oxide, zirconia, aluminium phosphates, titanium phosphates, zirconium phosphates, charcoal. The aluminates can also be chosen. It is preferable to use matrices containing alumina, in all its forms known to a person skilled in the art, and preferably gamma alumina.

The temperature of the reactor for carrying out the co-oligomerization is comprised between 40 and 600° C., preferably between 60 and 400° C. The pressure is comprised between 0.1 and 10 MPa, preferably between 0.3 and 7 MPa. The hourly space velocity is comprised between 0.01 and 100 h⁻¹ and preferably between 0.4 and 30 h⁻¹.

Advantageously, the addition of pentene-2 is carried out in such a way that the mass ratio of the predominantly linear oligomers to the pentene-2 is approximately 30/70 to 95/5, and preferably from 50/50 to 90/10, and even more preferably from 60/40 to 80/20.

The reactor can be of the fixed bed, tubular reactor, fluidized-bed or moving-bed type. It can be constituted by one or more beds with intermediate cooling.

For further details regarding the catalysts or the operating conditions, reference may be made to patent applications FR 2887555, EP 1299506 or EP0800568.

Separation of n-Pentane and iso-Pentane (Optional)

The n-pentane and isopentane optionally present in the effluent from the co-oligomerization can be removed in a separation step, for example by distillation.

### BRIEF DESCRIPTION OF THE DRAWINGS

FIG. 1 is a diagrammatic representation of the method of the invention.

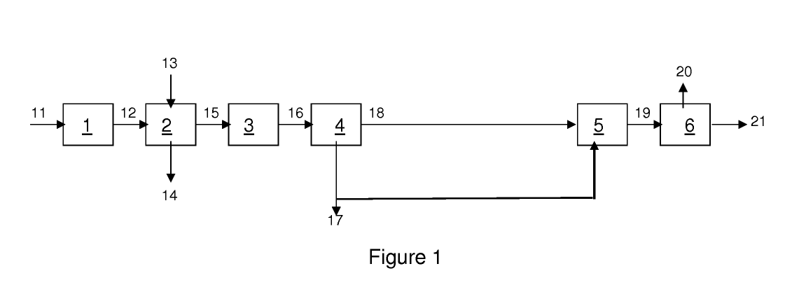

The C5 feedstock (11), which in addition to pentene-2 and pentene-1 comprises 2-methyl-2-butene, 2-methyl-1-butene, iso-pentane and n-pentane, pentadienes as well as traces of acetylene hydrocarbons, is subjected to an optional step of selective hydrogenation (1) of the pentadienes with isomerization of the alpha-olefins to internal olefins in thermodynamic equilibrium and hydrogenation of the acetylene hydrocarbons. This step thus makes it possible to remove the pentadienes and traces of acetylene hydrocarbons. The effluent (12) containing pentene-1, pentene-2, 2-methyl-2-butene, 2-methyl-1-butene, iso-pentane and n-pentane is then sent to an optional step (2) of separation of the isoamylenes (2-methyl-2-butene and 2-methyl-1-butene) by etherification in the presence of an alcohol (13) (methanol for example), making it possible to obtain a stream (14) containing TAME, and an effluent (15) containing pentene-1, pentene-2, n-pentene and iso-pentane. This effluent (15) is then sent to a step of selective oligomerization (3) of the pentene-1, making it possible to obtain an effluent (16) containing oligomers (mainly decenes) having a branching index less than or equal to 1, in a mixture with pentene-2, n-pentane and iso-pentane. A separation step (4), for example by distillation, then makes it possible to separate the oligomers formed (17) from a fraction (18) containing the unreacted pentene-2 in a mixture with n-pentane and iso-pentane. Optionally, a co-oligomerization (5) of the predominantly linear oligomers (17) can be carried out with the pentene-2 contained in fraction (18) to produce a fraction (19) containing pentadecenes, n-pentane and iso-pentane. The n-pentane and the iso-pentane (20) can be separated (6), for example by distillation, from the pentadecenes (21).

### EXAMPLES

The following examples illustrate the invention without limiting its scope.

Preparation of the Catalyst Precursor:

The catalytic composition used is prepared as follows. The bis(imino)pyridine ligand (1.58 g, 1.05 eq.) and the precursor iron(II) dichloride tetrahydrate (0.88 g, 1 eq.) are added to a Schlenk flask under argon containing a magnetic bar. 150 mL of THF is then added and the solution is stirred at room temperature for 16 hours. The solid formed is isolated by filtration and then washed with ether and with pentane (2.17 g, 91%).

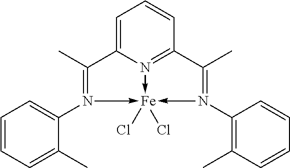

Catalytic Tests

The catalytic tests (Examples 1 to 2, Table 3) were carried out as follows: The 250-mL reactor is dried under vacuum at 80° C. for 3 hours, then placed under argon. The feedstock is injected into the reactor and then cooled down to 0° C. The iron complex (2.10⁻⁵ mol) in 3 mL of toluene is introduced as well as the activator (MAO, Al/Fe=105, 10 wt. % in toluene). Stirring is started and the temperature setting is fixed at 40° C. After the desired reaction time, neutralization is carried out with H₂O. The conversions are monitored by GC analysis of the gas and liquid phases.

In Example 1 (not according to the invention) the feedstock is pentene-1. In Example 2 (according to the invention), the feedstock is a mixture of pentene-1 and pentene-2 with a pentene-1/pentene-2 ratio of 0.83.

TABLE 3
Catalytic tests

| | | | | Dimers | Linear decenes | Branching index |
|---|---|---|---|---|---|---|
| | | Duration | Conversion of | (wt. %/total | (wt. % of n-decenes/ | of the dimers |
| No. | feedstock | (min) | pentene-2 | products) | total dimers) | obtained |
| 1 | pentene-1 | 120 | 0 | 93 | 48 | 0.48 |
| 2 | pentene-1 + | 120 | <6 | 96 | 44 | 0.44 |
| | pentene-2 | | | | | |

Example 2 shows that carrying out the method according to the invention makes it possible to separate pentene-2 from a C5 cut containing pentene-1 and pentene-2 by selective oligomerization of the pentene-1 to oligomers with a selectivity for dimers of 96 wt. % and a branching index in the dimers formed of less than 0.6. The conversion of the pentene-2 remains extremely low. Comparison of Examples 1 and 2 shows that the presence of pentene-2 has little effect on the selectivity and branching index.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding FR application No. 11/02.847, filed 20 Sep. 2011, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Method of separating pentene-2 from a C5 feedstock comprising pentene-1 and pentene-2, characterized by the following successive steps:
a step of selective oligomerization of pentene-1 to oligomers having a branching index less than or equal to 1 by an iron-based catalytic composition
a step of separation of pentene-2.

2. Method according to claim 1 in which the iron-based catalytic composition comprises at least one iron precursor and at least one ligand of the formula described below, complexed or not complexed with the iron precursor, said iron precursor can be hydrated or not and has the general formula FeXₙ, X being an anionic group chosen from a halide, a hydrocarbon group, a carboxylate, an oxide, an amide, an alkoxide, a hydroxide or a non-coordinating or weakly coordinating anion, said ligand having the general formula: 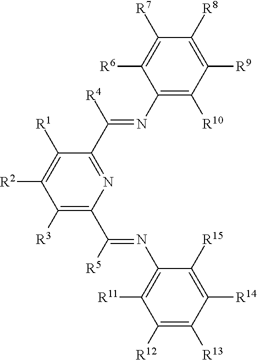
where R¹, R², R³, R⁴, R⁵, R⁶, R⁷, R⁸, R⁹, R¹⁰, R¹¹, R¹², R¹³, R¹⁴ and R¹⁵, which may be identical or different, are chosen from the hydrogen atom, linear or branched, cyclic or non-cyclic, saturated or unsaturated alkyl groups, aryl, aralkyl or alkaryl groups comprising 1 to 12 carbon atoms, groups containing hetero-elements, heterocyclic or not, aromatic or not, halides or not, supported or not.

3. Method according to claim 1 in which the catalytic composition comprises an activator chosen from aluminium derivatives and boron or zinc derivatives or a mixture of these derivatives.

4. Method according to claim 3 in which the activator is chosen from alkylaluminiums, alkylaluminium halides, aluminoxanes, trialkylboranes, tris(aryl)boranes, (aryl)borates associated with a triphenylcarbenium cation or with a trisubstituted ammonium cation, dialkylzincs.

5. Method according to claim 3 in which the activator is an aluminoxane chosen from methylaluminoxane (MAO) and/or ethylaluminoxane (EAO) and/or from the modified aluminoxanes such as modified methylaluminoxane (MMAO).

6. Method according to claim 1 in which the step of selective oligomerization is carried out in the presence of an organic solvent chosen from aliphatic or cyclic hydrocarbons, aromatic hydrocarbons, chlorinated solvents, acetonitrile, diethyl ether and/or tetrahydrofuran, and/or a liquid ionic solvent.

7. Method according to claim 1 in which the step of selective oligomerization is carried out at a temperature between −40 to +250° C. and at a pressure varying from atmospheric pressure to 10 MPa.

8. Method according to claim 1 in which the feedstock originates from a catalytic cracking unit and/or a steam cracking unit.

9. Method according to claim 1 in which the feedstock also contains 2-methyl-2-butene and 2-methyl-1-butene and a step of separation of 2-methyl-2-butene and 2-methyl-1-butene is carried out before the step of selective oligomerization by etherification in order to produce alkyl tert-butyl ethers.

10. Method according to claim 9 in which the etherification is carried out in the presence of an etherification catalyst with an alcohol the hydrocarbon chain of which comprises from 1 to 10 carbon atoms.

11. Method according to claim 9 in which a step of co-oligomerization of oligomers having a branching index less than or equal to 1 obtained by selective oligomerization with pentene-2 is carried out in the presence of an acid catalyst after the step of selective oligomerization.

12. Method according to claim 11 in which the co-oligomerization is carried out in the presence of an acid catalyst at temperatures between 40 and 600° C. and at pressures ranging from 0.1 MPa to 10 MPa.

13. Method according to claim 1 in which the feedstock also contains pentadienes and optionally traces of acetylene hydrocarbons and a step of separation of the pentadienes and any traces of acetylene hydrocarbons is carried out by selective hydrogenation before the step of separation of 2-methyl-2-butene and of 2-methyl-1-butene by etherification and/or the step of selective oligomerization.
